(19) United States
(12) Patent Application Publication
(10) Pub. No.: US 2024/0226524 A1
(43) Pub. Date: Jul. 11, 2024

(54) Venous Access Port with Molded and/or Radiopaque Indicia

(71) Applicant: Medical Components, Inc., Harleysville, PA (US)

(72) Inventors: Timothy M. Schweikert, West Chester, PA (US); Raymond R. Bizup, Feasterville, PA (US); Kevin E. Sanford, Chalfont, PA (US); Kenneth M. Zinn, Westport, CT (US)

(21) Appl. No.: 18/615,101

(22) Filed: Mar. 25, 2024

Related U.S. Application Data
(63) Continuation of application No. 17/865,449, filed on Jul. 15, 2022, now Pat. No. 11,938,296.
Application No. 17/865,449 is a continuation of application No. 16/035,879, filed on Jul. 16, 2018, now Pat. No. 11,406,808.
Application No. 16/035,879 is a division of application No. 15/350,273, filed on Nov. 14, 2016, now Pat. No. 11,478,622.
Application No. 15/350,273 is a continuation of application No. 13/550,745, filed on Jul. 17, 2012, now Pat. No. 9,533,133.
Application No. 13/550,745 is a continuation of application No. 12/143,377, filed on Jun. 20, 2008, now Pat. No. 8,257,325.
(60) Provisional application No. 60/936,491, filed on Jun. 20, 2007.

(51) Int. Cl. A61M 39/02; A61M 5/00

(52) U.S. Cl. CPC A61M 39/0208; A61M 5/007; A61M 2039/0205; A61M 2039/0229; A61M 2039/0238; A61M 2205/32

(57) ABSTRACT

A venous access port assembly having a base, a peripheral surface, and a septum. The base defines an interior reservoir. The peripheral surface includes integrally molded X-ray discernable indicia identifying that the assembly is rated for power injection. The X-ray discernable indicia may extend through a height of the peripheral surface from a top surface to a bottom surface thereof. According to one aspect, the peripheral surface may be formed from X-ray discernable material, and the X-ray discernable indicia may be formed from the X-ray discernable material, or they may be formed by voids in the X-ray discernable material. According to another aspect, the peripheral surface may be formed from a radiotransparent or radiolucent material and applied with a radiopaque agent, and the X-ray discernable indicia may be one or more voids in the radiopaque agent or may be portions of the peripheral surface applied with the radiopaque agent.

### RELATED APPLICATIONS

This application is a continuation of, and claims the benefit of, co-pending U.S. patent application Ser. No. 17/865,449, filed Jul. 15, 2022, which is a continuation of U.S. patent application Ser. No. 16/035,879, filed Jul. 16, 2018, now U.S. Pat. No. 11,406,808, which is a divisional of U.S. patent application Ser. No. 15/350,273, filed Nov. 14, 2016, now U.S. Pat. No. 11,478,622, which is a continuation of U.S. patent application Ser. No. 13/550,745, filed Jul. 17, 2012, now U.S. Pat. No. 9,533,133, which is a continuation of U.S. patent application Ser. No. 12/143,377, filed Jun. 20, 2008, now U.S. Pat. No. 8,257,325, which claims the benefit of U.S. provisional application No. 60/936,491, filed Jun. 20, 2007, which applications are incorporated herein by reference.

### FIELD OF THE INVENTION

This invention relates generally to medical devices, and more particularly to venous access ports having integrally molded and/or radiopaque indicia.

### BACKGROUND OF THE INVENTION

Venous access ports for the infusion and/or withdrawal of fluids from a patient are well-known, secured to the proximal end of an implanted catheter. These ports are typically used for drug infusion or for withdrawal of small amounts of blood, where large flows of fluid are not required. The ports are assemblies of a needle-impenetrable housing with a discharge port in fluid communication with a catheter and a reservoir within the port housing, and provide as subcutaneous self-sealing septum that defines an access site for multiple needle sticks through the covering skin tissue of the patient, through the septum, and into the reservoir, without the need to continuously search for new access sites. Examples of such ports are disclosed, for example, in U.S. Pat. Nos. 4,704,103; 4,762,517; 4,778,452; 5,185,003; 5,213,574; and 5,637,102.

It is desired to provide a venous access port assembly that provides a medical practitioner with the capability to discern an important property of the port assembly after the port assembly has been implanted into a patient.

### BRIEF SUMMARY OF THE INVENTION

The present invention is directed to an implantable venous access port having a marking visible by X-ray examination, providing identification on the X-ray of characteristics of the implanted access port. These characteristics might otherwise be unknown once the port is implanted under the skin of the patient. Considering that X-ray examination is normal practice prior to medical access of the port, an X-ray examination that also identifies important characteristics of an implanted port is beneficial to the practitioner.

Accordingly, one aspect of the present invention is the incorporation of a molded marking into or onto the venous access port, or the addition of radiopaque indicia onto the access port, and/or the addition of radiopaque indicia onto a molded marking integrated into the access port, all of which would be discernable under X-ray examination to provide information concerning a characteristic or attribute of the venous access port, so that a practitioner, subsequent to implantation of the access port under the skin of a patient, can determine that characteristic or attribute of the port by X-ray examination.

One such characteristic could be power injectable capability; that is, an indication that the venous access port is rated for the power injection of contrast fluid. Power injection capability can be indicated with the letters “CT,” for “computed tomography,” or “contrast enhanced computed tomography.” “CT” would indicate the access port's capability to withstand high pressures used during injection of contrast fluid into a patient, and the letters “CT” would be understood in medical practice to indicate that the port is suitable for the high pressure injection of contrast fluid. Naturally, any characteristic or attribute of the venous access port could be indicated, and any choice of letters or symbols could be employed.

In one embodiment, an access port of the present invention includes a housing and a septum, providing an interior reservoir and a passageway extending from the reservoir through a stem of a discharge port to establish fluid communication with a proximal end of a catheter lumen to which the port assembly is secured prior to placement of the assembly into a patient. The port may optionally have more than one reservoir and associated septum. The housing could include molded therein the letters “CT.” If the housing is made of a plastic material, such as a silicone elastomer the letters “CT” (or the entire housing) could be loaded with a radiopaque agent, allowing the letters “CT” (or the cadre housing to be seen by X-ray examination. If the housing comprises a metal material, or if the portion of the housing including the letters “CT” is made of a metal material, the letters would naturally be visible by X-ray examination.

### BRIEF DESCRIPTION OF THE DRAWINGS

The accompanying drawings, which are incorporated herein and constitute part of this specification, illustrate the presently preferred embodiments of the invention, and together with the general description given above and the detailed description given below, serve to explain the features of the invention. In the drawings;

FIG. 1 is an isometric view of one embodiment of a venous access port of the present invention;

FIG. 2 is a pan view of the port FIG. 1;

FIGS. 3 and 4 are cross-sectional views of the port of FIGS. 1 and 2 taken along lines 3-3 and lines 4-4 of FIG. 2, respectively;

FIG. 5 is an isometric view of a needle-impenetrable housing base of the venous access port of FIG. 1;

FIGS. 6 and 7 are transverse cross-sectional and longitudinal cross-sectional views of the housing base of FIG. 5;

FIG. 8 is an isometric view from below of one embodiment of the housing base of FIG. 5, showing radiopaque indicia applied on the housing base bottom surface;

FIGS. 9 and 10 are bottom and top views of the housing base of FIG. 8 having radiopaque indicia thereon, with the top view (FIG. 10) being analogous to a X-ray view of the venous access port seen by a radiologist, with indicia visible by X-ray shown by dashed lines in FIG. 10;

FIG. 11 illustrates top views of two alternative embodiments of the needle-impenetrable housing base of the venous access port of FIG. 1, showing integrally molded “CT” markings;

FIG. 12 illustrates an isometric view of one of the FIG. 11 embodiments of the needle-impenetrable housing base of the venous access port of FIG. 1, featuring an integrally molded “CT” marking where the letters “CT” are formed of housing material; and

FIG. 13 illustrates an isometric view of the other of the FIG. 11 embodiments of the needle-impenetrable housing base of the venous access port of FIG. 1, featuring an integrally molded “CT” marking where the letters “CT” comprise voids in the housing material, as if cut or punched out of the housing material.

### DETAILED DESCRIPTION OF THE INVENTION

Certain terminology is used herein for convenience only and is not to be taken as a limitation on the present invention. The terms “distal” and “proximal” refer, respectively, to directions closer to and away from the insertion tip of a catheter in an implantable catheter assembly. The terminology includes the words specifically mentioned, derivatives thereof, and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

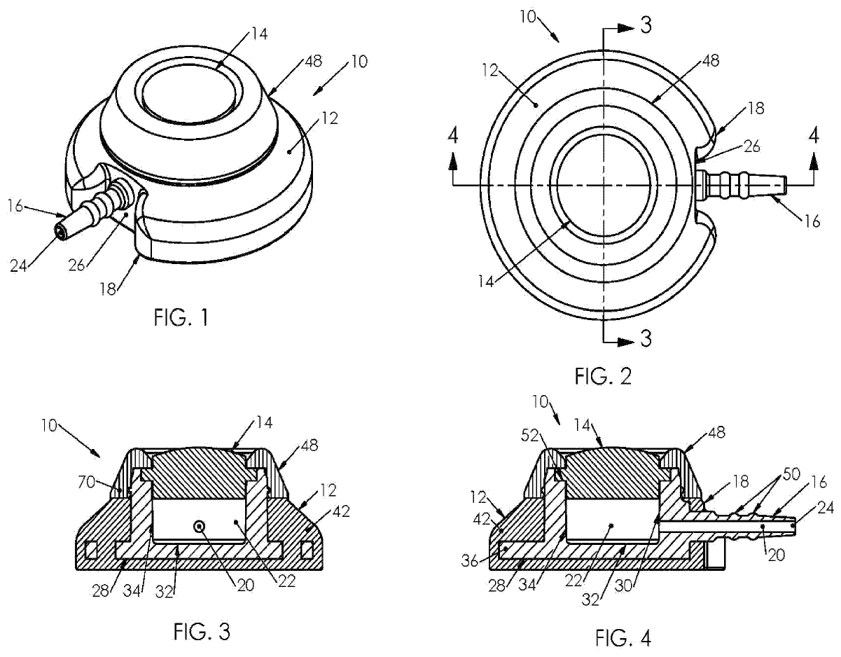

Venous access port assembly 10 of FIGS. 1 to 4 includes a housing 12 and a septum 14, with a discharge port 16 extending from a distal end 18 of the port assembly 10 to be attached securely and sealingly to the proximal end of a catheter (not shown). A passageway 20 extends from the interior reservoir 22 to the distal tip opening 24 of discharge port 16. A recess 26 is seen to be provided along both sides of discharge port 16, facilitating insertion of the discharge port 16 into the catheter lumen and providing a clearance for a locking sleeve or clamp (not shown) utilized to compress the catheter lumen wall against the exterior surface of the discharge port 16 for assured sealed connection of the catheter with the port assembly 10.

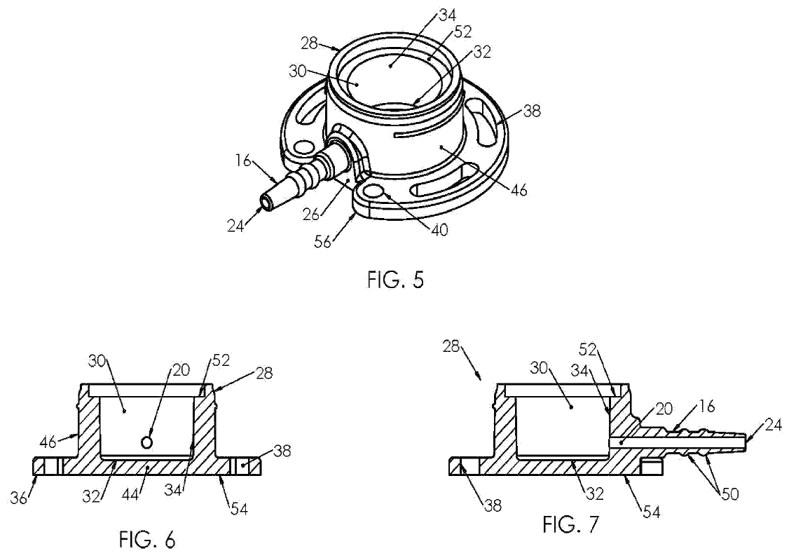

With reference now to FIGS. 3 to 7, the interior of the port assembly 10 is shown to provide an interior reservoir 22. Housing 12 is shown to include a housing base 28 of needle-impenetrable material that includes a web 30 having a bottom boor 32 and side walls 34 that define the interior reservoir 22 beneath septum 14. Bottom floor 32 may be convex or elevated (not shown) toward the center of the reservoir, if desired. Housing base 28 includes a base flange 36 extending radially outwardly from the bottom of well 30, and base flange 36 includes openings 38, 40 that serve to enable suturing to the patient upon placement of the venous access port and the attached catheter into the patient.

As shown in FIGS. 3 and 4, a skirt 42 is overmolded about housing base 28 and may be of silicone elastomer. It is seen that skirt 42 encapsulates the outer surfaces of the bottom wall 44 and the bottom portion of the side walls 46 of housing base 28, and is shown to fill in the suture holes 38, 40; but since the material is silicone elastomer, suturing is possible since the suturing needle can easily be inserted through the material of skirt 42 and through the suture holes, and thereafter the filled openings provide minimal opportunity for ingrowth of patient tissue into the openings.

Also seen in FIGS. 1 to 4 is cap 48, which secures to housing base 28 to in turn secure septum 14 in position in the port assembly 10. Preferably, skirt 42 is insert molded onto base flange 36 of housing base 28 after cap 48 is secured to the upper portion of housing base 28 to secure the septum in position. It is seen in FIGS. 4 and 7 that discharge port 16 is integral with housing base 28 as is preferable. Discharge port 16 is shown to have a pair of annular ridges 50 that facilitate with the mechanical connection of the catheter proximal end with the port assembly 10. Housing base 28 includes a septum seat 52 extending into the top of well 30, into which a flange of the septum will be seated, preferably under radially inward compression. Housing base 28 has a bottom outer surface 54.

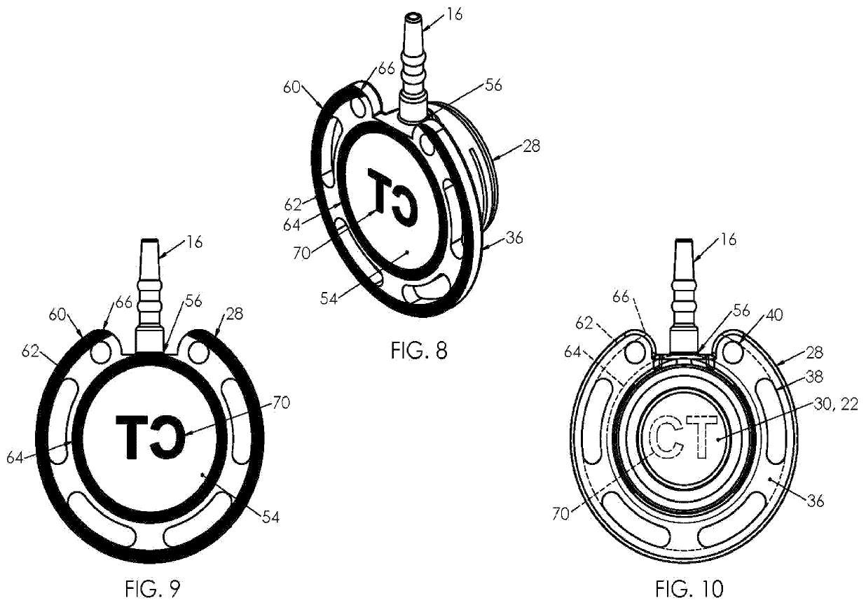

Radiopaque markings 60 of the present invention are shown in FIGS. 8 to 10. A larger outer circle 62 is seen provided on the outermost periphery of bottom base surface 54, and a smaller inner circle 64 is seen provided within the area circumscribed by the suture openings 38 and holes 40 through base flange 36. Adjacent to discharge port 16, a recess 56 is provided in the skirt of the housing base to provide a clearance for use of a connection sleeve that will be used to secure the catheter (not shown), and outer circle 62 is shown to have a gap 66 at the recess. Outer and inner circles or rings 62, 64 circumscribe radiopaque indicia 70.

Radiopaque indicia 70 are provided on bottom outer surface 54 within the region directly beneath the reservoir and septum. In the example shown, indicia 70 comprise the letters “CT” (FIG. 10) representing the term “computed tomography.” The meaning of this particular example of indicia is that the venous access port assembly 10 is rated for high pressure injection such as is necessary for infusion into the patient of contrast medium that is used in computed tomography. Other indicia may of course be used that indicate some other attribute or characteristic of the venous access port assembly. The radiopaque markings and indicia would appear on an X-ray of the patient, and the indicia are provided in a mirror-image orientation on the bottom outer surface of the housing base (FIGS. 7 and 8) so that the indicia would appear as “CT” when the X-ray is viewed (FIG. 9), easily discerned by the radiologist or technologist. Centering of the indicia within the region (identified as “30, 22” in FIG. 10) directly beneath the reservoir and septum minimizes any obscuring by the structure of the venous access port assembly, and the indicia may also be easily discernable should the port assembly be at an angle from the horizontal plane of the X-ray; the outer and inner circles 62, 64 would appear oval or elliptical should the port assembly be at such an angle. Gap 66 in outer circle 62 would also appear and would indicate the location of the discharge port stem 16,

The radiopaque markings may constitute marking fluid that is embossed or imprinted or otherwise applied onto the surface of the housing base 28, such as black radiopaque ink Part No. C11002 Rev A formulated by Creative Imprinting of Erie, Pa., from Marabu Tampapur TPU 910 clear with tungsten added, available from Marabuwerke GmbH & Co. KG of Stuttgart, Germany, and may be applied on plasma-treated surfaces. At least the housing base 28, the septum 14 and the skirt 42 are of radiotransparent or radiolucent material as is well known in implanted medical devices, and the housing base may be molded of polysulfone resin.

The radiopaque markings may alternatively applied to the inwardly facing surface of the bottom wall of the housing base, or may constitute foil or film (such as a decal) of radiopaque material embedded within the housing base, these alternatives not being shown in the drawings.

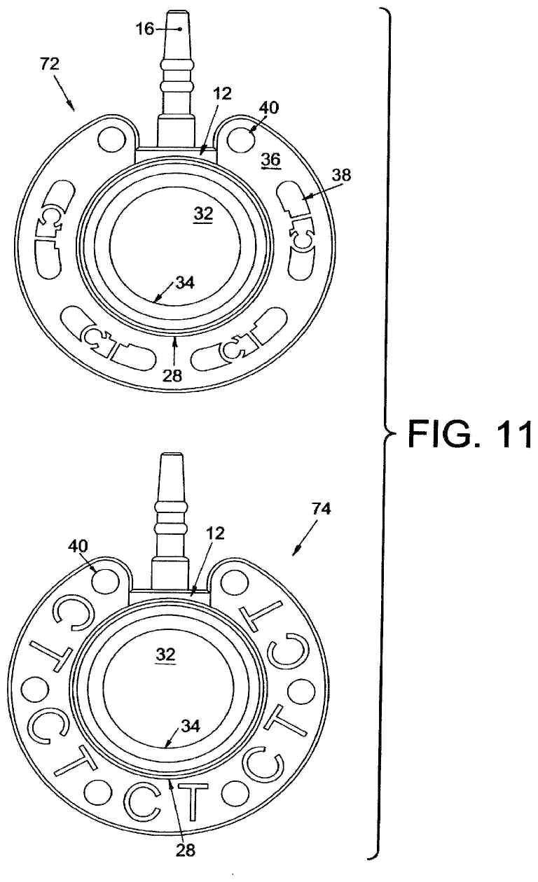

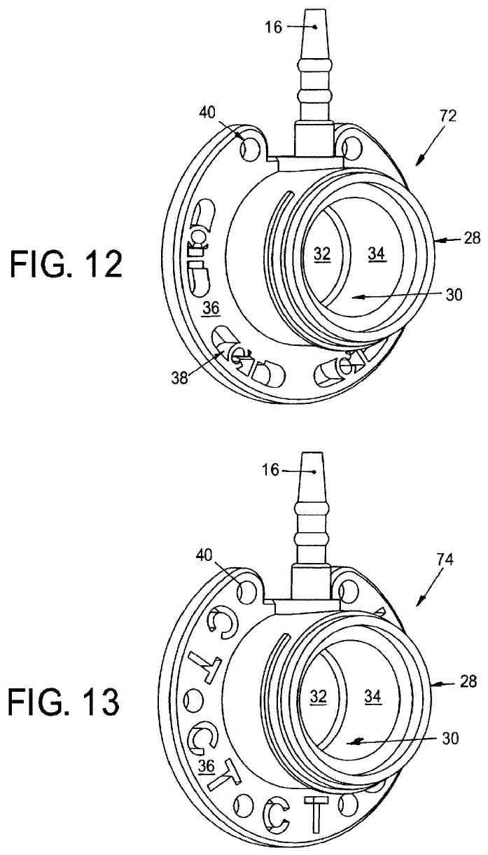

FIG. 11 illustrates top views of two alternative embodiments 72, 74 of the needle-impenetrable housing base 28 of the venous access port 10 of the present invention. Each of the alternative embodiments 72, 74 shows “CT” markings integrally formed in the base flange 36 of the housing base 28. The first alternative embodiment 72, an isometric view of which is illustrated in FIG. 12, features integrally molded “CT” markings within the suture openings 38, where the letters “CT” are formed of the base flange 36 material. The second alternative embodiment 74, an isometric view of which is illustrated in FIG. 13, features integrally molded “CT” markings alongside the suture openings 38, 40, where the markings are voids in the base flange 36 material, as if cut or punched out of the base flange 36 material. Naturally, the markings could be configured elsewhere within or about the housing base 28, or within a peripheral portion of another component of the venous access port 10.

If the housing base 28 is made of a plastic material, such as a silicone elastomer or polysulfone resin, the letters “CT,” or the base flange 36, or the entire housing base 28, could be applied with a radiopaque agent or fluid, allowing the applied area to be visible by X-ray examination. If the housing base 28 is comprised of a metal material, or if the base flange 36, or a portion thereof, is comprised of metal (particularly the letters “CT”), those portions would naturally be visible by X-ray examination without application of the radiopaque agent.

It will be appreciated by those skilled in the art that changes could he made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A venous access port assembly for implantation into a patient, comprising:
a needle-penetrable septum;
a housing comprising a housing base at least partially defining at least one reservoir; and
a bottom surface comprised at least partially of a radiopaque material,
the bottom surface comprising one or more X-ray discernable indicia at least partially cut or punched out of the radiopaque material,
wherein the one or more X-ray discernable indicia indicating under X-ray examination that the venous access port assembly is capable of withstanding pressures used during injection of contrast fluid.

2. The venous access port assembly of claim 1, wherein the bottom surface includes at least one of a bottom surface of the housing base, or a bottom surface of a base flange.

3. The venous access port assembly of claim 2, wherein the bottom surface of the housing base comprised of at least partially the radiopaque material.

4. The venous access port assembly of claim 3, wherein the one or more X-ray discernable indicia are in the bottom surface of the housing base.

5. The venous access port assembly of claim 2, wherein the bottom surface of the base flange comprised of at least partially the radiopaque material.

6. The venous access port assembly of claim 5, wherein the one or more X-ray discernable indicia are in the bottom surface of the base flange.

7. The venous access port assembly of claim 6, wherein the one or more X-ray discernable indicia extend completely through the radiopaque material.

8. The venous access port assembly of claim 1, wherein the housing further comprises a cap.

9. The venous access port assembly of claim 1, further comprising a radiotransparent or radiolucent skirt overmolded about at least a portion of the housing.

10. The venous access port assembly of claim 1, wherein the one or more X-ray discernable indicia indicate an orientation of the venous access port assembly after implantation.

11. The venous access port assembly of claim 1, wherein the one or more X-ray discernable indicia are shaped as letters or symbols capable of indicating an orientation of the venous access port assembly after implantation.

12. The venous access port assembly of claim 1, wherein the one or more X-ray discernable indicia are in the shape of letters “CT”.
